(19) United States
(12) Patent Application Publication
(10) Pub. No.: US 2024/0093155 A1
(43) Pub. Date: Mar. 21, 2024

(54) METHOD OF CHANGING CULTURE MEDIUM OF A CULTURE USING SPINFILTERS

(71) Applicants: SARTORIUS STEDIM BIOTECH GMBH, Göttingen (DE); REPAIRON GMBH, Göttingen (DE)

(72) Inventors: Luis HAUPT, Göttingen (DE); Julia HUPFELD, Adelebsen (US)

(73) Assignees: SARTORIUS STEDIM BIOTECH GMBH, Göttingen (DE); REPAIRON GMBH, Göttingen (DE)

(21) Appl. No.: 18/262,433

(22) PCT Filed: Jan. 21, 2022
(86) PCT No.: PCT/EP2022/051314

§ 371 Date: Jul. 21, 2023

(30) Foreign Application Priority Data
Jan. 21, 2021 (EP) 21152729.6

(51) Int. Cl. C12N 5/074; C12M 1/00; C12M 1/06

(52) U.S. Cl. CPC C12N 5/0696; C12M 27/06; C12M 29/04; C12M 29/10

(57) ABSTRACT

The present invention relates to a method of expanding stem cells cultured as cell aggregates in a suspension culture changing culture medium and a method of medium exchange for the same cells characterized in the use of a rotating mesh such as a spinfilter device. The present invention further relates to a use of a rotating mesh for medium exchange in a suspension culture of stem cells.

### CROSS-REFERENCE TO RELATED APPLICATIONS

The present application claims the benefit of priority of European Patent Application No. 21 152 729.6 filed 21 Jan. 2021, the content of which is hereby incorporated by reference it its entirety for all purposes.

### TECHNICAL FIELD OF THE INVENTION

The present disclosure relates to a method of expanding stem cells cultured as cell aggregates in a suspension culture changing culture medium and a method of medium exchange characterized in the use of a rotating mesh such as a spinfilter device. The present disclosure further relates to a use of a rotating mesh for medium exchange in a suspension culture of stem cells.

### BACKGROUND

In basic research, with a lower demand for large amounts of cells, PSCs, iPSCs and iPSC-derived cells are routinely grown as adherent cell culture. Here, the cells attach to the surface of a culture dish and grow as colonies or a monolayer. The adherent cell culture of iPSCs however is not suitable for the generation of large amounts of cells that are needed for clinical applications. This is because it is material- and labor-intensive. Furthermore, the outcome and quality of the cell production highly depends on the operator, because the process is usually not automated and only poorly monitored and controlled.

It has been reported that the use of bioreactor systems enables production of large amounts of PSCs, iPSCs and iPSC-derived cells (Kropp et al., 2017). In these systems, iPSCs and iPSC-derived cells usually do not attach to the surface of a dish but are grown in a free-floating suspension because PSCs form aggregates when cultivated in suspension. Suspension culture in bioreactor systems is described to be more efficient than adherent culture because the culture can be monitored, controlled and automated even at high cell numbers and less material and amount of work is needed. Importantly, for these reasons the use of bioreactor systems would be preferred over static culture for GMP-controlled applications. Different bioreactor systems have been reported for suspension culture of PSCs with stirred tank reactor (STR) systems being the best described ones. It was shown that high numbers of iPSCs and iPSC-CMs can be successfully generated in STR systems (Chen et al., 2012; Halloin et al., 2019; Hemmi et al., 2014; Jiang et al., 2019; Kempf et al., 2015; Kropp et al., 2016).

Despite the advantages of bioreactor systems for large-scale PSC, iPSC and iPSC-CM production, the suspension culture creates several new challenges. For instance, the exchange of culture medium in a suspension culture is more elaborate than in an adherent cell culture. This is because PSCs have to be retained in the culture during removal of spent medium to prevent cell loss. Repeated batch feeding strategies are often described in STRs for the medium exchange (Kropp et al., 2017). Here, the agitation is stopped and cell aggregates settle to the bottom of the vessel. Subsequently, the medium is discarded without disturbing the settled aggregates. Fresh medium is added and agitation is continued. This strategy may cause fusion of settled aggregates and thereby spontaneous differentiation of iPSCs. The degree of aggregate fusion depends on the duration without agitation. Especially in larger systems, the repeated batch feeding strategy will likely cause high amounts of fused aggregates because the settling time increases with the height of the vessel and it may also take more time to exchange larger volumes of medium. Furthermore, the one-time exchange of a large amount of medium causes a sudden change of culture parameters such as pH, oxygen concentration and concentrations of metabolites, nutrients and signaling factors. This may cause additional stress for the PSCs resulting in reduced proliferation.

Accordingly, there is still a need for methods of changing culture medium or expansion of cells of a suspension culture comprising stem cells, in particular in which the entire process can be performed without removing the cells or aggregates from the system and without exposing the cells to the stress that is associated with sedimentation and/or centrifugation for extended periods of time. The present invention aims to address this need.

### SUMMARY OF THE INVENTION

This problem is solved by the subject-matter as defined in the claims. It is presented herein a method of expanding stem cells, wherein the stem cells are comprised in cell aggregates in a suspension culture, a method of changing culture medium of a suspension culture, the suspension culture comprising cell aggregates of stem cells suspended in the culture medium, and a use of a rotating mesh as defined herein for medium exchange in a suspension culture, the suspension culture comprising cell aggregates suspended in the culture medium.

Accordingly, the present invention relates to a method of expanding stem cells, wherein the stem cells are comprised in cell aggregates in a suspension culture, the method comprising:
(i) culturing the stem cells under conditions that allow proliferation of the stem cells; and
(ii) performing medium exchange by perfusion through a rotating mesh.

The present invention further relates to a method of changing culture medium of a suspension culture, the suspension culture comprising cell aggregates of pluripotent stem cells suspended in the culture medium, the method comprising:
(i) performing medium exchange by perfusion through a rotating mesh; and
(ii) optionally replacing the medium removed through the rotating mesh with fresh medium.

The present invention further relates to the use of a rotating mesh as defined herein for medium exchange in a suspension culture, the suspension culture comprising cell aggregates suspended in the culture medium, wherein the cells are stem cells.

The cells may be cultured in a bioreactor, wherein the bioreactor preferably is a stirred bioreactor, a rocking motion bioreactor and/or a multi parallel bioreactor.

The medium exchange may be performed inside a bioreactor.

The rotating mesh may be a spin-filter, optionally wherein the spin-filter is attached to the stirrer or stirring rod of a bioreactor.

The medium exchange may be performed outside of a bioreactor, preferably wherein the device housing the rotating mesh is fluidly coupled with the bioreactor to form a closed system.

The rotating mesh may have a pore size of about 1 μm to about 50 μm, of about 5 μm to about 50 μm, of about 10 μm to about 50 μm, of about 5 μm to about 40 μm, about 5 μm to about 30 μm, about 5 μm to about 20 μm, or about 5 μm to about 15 μm, preferably about 10 μm.

The cell aggregates may have an average diameter between about 50 and about 300 μm, between about 80 and about 250 μm, between about 100 and about 220 μm or between about 100 μm to about 200 μm.

The stem cells may be pluripotent stem cells, cord blood stem cells, mesenchymal stem cell and/or hematopoietic stem cells; and/or cells derived from stem cells. The pluripotent stem cells preferably are induced pluripotent stem cells (iPSC), embryonic stem cells (ESC), parthenogenetic stem cells (pPSC) or nuclear transfer derived PSCs (ntPSC), most preferably iPSCs. The stem cells preferably are selected from the group consisting of TC-1133, the Human Episomal iPSC Line of Gibco ATCC ACS-1004, ATCC ACS-1021, ATCC ACS-1025, ATCC ACS-1027, ATCC ACS-1030.

### BRIEF DESCRIPTION OF THE DRAWINGS

The invention will be better understood with reference to the detailed description when considered in conjunction with the non-limiting examples and the accompanying drawings, in which:

FIG. 1 shows light microscopy images of iPSC suspension culture and the discarded medium, which was aspirated using a rotating mesh, here exemplarily a spinfilter, as a cell retention device. FIG. 1A shows a sample of the suspension culture of passage 0 while FIG. 1B shows a sample of the discarded medium of the same passage. FIG. 1C shows a sample of the aggregate suspension culture of passage 1, wherein aggregates present variable dimensions, while FIG. 1D shows a sample of the discarded medium of the same passage demonstrating the efficient filter capacity. Scale bars: 400 μm.

FIG. 2 shows the aggregate size of PSC cell aggregates in two different UniVessel sizes (0.5 L and 2 L), which were perfused with a rotating mesh, at various days of culturing.

FIG. 3 shows the expression of pluripotency-related genes in iPSCs at day 4 of passage 0 cultured in the UniVessel 2 L (vessel 2), which were perfused with a rotating mesh.

FIG. 4 shows the expression of pluripotency-related genes in iPSCs at day 4 of passage 0 cultured in the UniVessel 0.5 L (vessel 3), which were perfused with a rotating mesh.

### DETAILED DESCRIPTION OF THE INVENTION

The present invention is described in detail in the following and will also be further illustrated by the appended examples and figures.

Automated medium exchange of suspension cultures, especially suspension cultures of stem cell aggregates, in a bioreactor remains a challenge. Manual medium exchange usually involves the transfer of at least a portion of the suspension culture out of the bioreactor and includes, e.g., centrifugation of the cells. This mechanical stimulation can have negative effects on cell viability or functions such as unwanted differentiation of stem cells (Lipsitz et al. 2018). One further possibility of automated medium exchange of a suspension culture in a bioreactor (“vessel settling”) is stopping of the stirring and allowing the cells to settle at the bottom of the bioreactor. The supernatant can then be aspirated and be replaced with fresh medium. This however also leads to mechanical stimulation of the cells, which can lead to irregular growth and loss of pluripotency. This problem is overcome by the method of the present invention:

The use of a rotating mesh, such as a spinfilter cell retention device, allows for perfusion medium exchange with minimal interference with the cell culture, which is especially desirable for a GMP-guided process. The spent medium can be separated from the stem cell aggregates directly in the culture vessel, surprisingly without disturbing the stem cells in any way. In contrast, the application of other cell retention devices often requires the cell suspension to be transferred out of the culture vessel. Such a removal from the culture vessel likely causes a decrease in stem cell quality due to increased shear stress, fusion of aggregates and short-term alterations in the cell environment. Furthermore, external devices need to be operated and are an additional source of error during the process. The application of a microsparger as cell retention device has been described and similar advantages as explained above have been proposed. However, microspargers are not designed to be applied as cell retention devices and may easily clog, thereby causing the failure of the suspension culture. This is because the surface of the microsparger is small and the filter sits directly in front of the aspiration tube. Furthermore, the aggregates in a suspension culture may actively attach to the static microsparger once they got aspirated to it. On the other hand, the risk of clogging of a spin filter is little, because of its high surface area. The spinning motion of a spinfilter device further reduces the risk of clogging.

As shown in Examples 1 and 2, the application of a rotating mesh as cell retention device allows maintaining cell aggregate of pluripotent stem cells in perfect shape while at the same time debris and dead cells can easily be removed. Due to the sensitivity of stem cells to shear stress as described above, it was a surprise that also stem cell aggregates can be cultured in a perfusion suspension culture using a rotating mesh for medium exchange without harming the stem cells.

Accordingly, the present invention relates to a method of expanding stem cells, wherein the stem cells are comprised in cell aggregates in a suspension culture, the method comprising:
(i) culturing the stem cells under conditions that allow proliferation of the stem cells; and
(ii) performing medium exchange by perfusion through a rotating mesh.

The present invention further relates to a method of changing culture medium of a suspension culture, the suspension culture comprising cell aggregates of stem cells suspended in the culture medium, the method comprising:
performing medium exchange by perfusion through a rotating mesh; and
(ii) optionally replacing the medium removed through the rotating mesh with fresh medium.

Perfusion is characterized by the continuous replacement of medium from the reactor by fresh medium while retaining cells in the vessel by specific systems. Perfusion is an operation mode for biopharmaceutical production processes enabling highest cell densities and productivity. Beside the advantage that cells in perfusion are constantly provided with fresh nutrients and growth factors, potentially toxic waste products are washed out, ensuring more homogeneous conditions in the reactor. Moreover, compared to repeated batch processes, perfusion processes support process automation and improved feedback control of the culture environment, including DO (dissolved oxygen), pH, and nutrient concentrations. Perfusion cultures may enable a relatively stable, physiological environment that also supports the self-conditioning ability of PSCs by their endogenous factor secretion and thus eventually reducing supplementation of expensive medium components. In sum, a perfused culture leads to higher yields and quality of the cells.

A “rotating mesh” as used herein relates to a cell retention device. The rotating mesh is characterized by the presence of openings that allow the flow of spent medium including debris such as dead cells out of the suspension culture but retains the cell aggregates in the culture vessel. This is also the principle of perfusion culture. Thereby, the “used” medium can flow out of the bioreactor. The outflow can be compensated by an inflow of medium, preferably at a rate that essentially equals the outflow, thereby maintaining optimal growth conditions for the suspension culture for an extended period of time. The rotating mesh often is in the form of a cylinder, wherein usually the side but sometimes also the top and/or the bottom contains the openings. The rotating mesh may be attached to the stirrer or stirring rod of a bioreactor. One example of a rotating mesh described herein is a spinfilter. The rotating mesh may be made from any suitable material such as a plastic or metal. Preferred the rotating mesh is made of stainless steel. The rotating mesh preferably is autoclavable but also can be provided in form of a (pre-sterilized) single-use rotating mesh.

The rotating mesh divides the culture vessel, into two compartments, an “inside” compartment that contains the cell aggregates suspended in culture medium and an “outside” compartment. In the context of a bioreactor, in which a rotating mesh is placed on the stirrer, “outside” is the inner compartment of the rotating mesh, from which the used medium is removed, while the “inside” compartment means that part of the culture vessel, which is outside of the rotating mesh. The inside compartment advantageously is designed to allow an outflow of used media. Exemplary rotating meshes include spinfilters. Spinfilters are known to a person skilled in the art and, e.g., be described in WO 92/05242. The rotating mesh may be mounted on the impeller of a bioreactor. In this case, the rotating mesh has the same rotational speed as the impeller of the bioreactor. A person skilled in the art is capable of determining a suitable rotational speed that is suitable for both, growth of stem cells and perfusion of the culture medium through the rotating mesh. Typical impeller rotational speeds include 85 to 140 rpm.

The pore size of the rotating mesh preferably is chosen to allow retention of the cell aggregates while at the same time used culture medium including (cell) debris can pass through or “perfuse” the rotating mesh. The optimal pore size may vary with the cell type cultured. In some examples, the rotating mesh may have a pore size of about 1 μm to about 50 μm, of about 5 μm to about 50 μm, of about 10 μm to about 50 μm, of about 5 μm to about 40 μm, about 5 μm to about 30 μm, about 5 μm to about 20 μm, or about 5 μm to about 15 μm, preferably about 10 μm. In this context it is worth noting that when a suspension culture of stem cells is started (e.g., in a bioreactor), the cells may be present as single cells or only small cell aggregates that are not retained by the rotating mesh. During the initial growth phase, when also the demand for nutrients is still low, it might be advisable not to start any liquid flow through the rotating mesh and out of the culture device such as a bioreactor to avoid loss of stem cells before the cell aggregates have reached a size that is retained in the suspension culture by the rotating mesh.

The term “suspension culture” as used herein is a type of cell culture in which single cells or small aggregates of cells are allowed to function and multiply in an preferably agitated growth medium, thus forming a suspension (c.f. the definition in chemistry: “small solid particles suspended in a liquid”). This is in contrast to adherent culture, in which the cells are attached to a cell culture container, which may be coated with proteins of the extracellular matrix (ECM). In suspension culture, in one embodiment no proteins of the ECM are added to the cells and/or the culture medium. The suspension culture preferably is essentially free of solid particles such as beads, microspheres, microcarrier particles and the like; cells or cell aggregates are no solid particles within this context. In one embodiment, the cells are not in microcarrier (suspension) culture.

“Expansion” or “cell expansion” and also “cell proliferation” as used herein relate to an increase in the number of cells as a result of cell growth and cell division.

In methods of the invention, may it be the method of expanding stem cells or the method of changing culture medium of a suspension culture, the cells may be cultured in a bioreactor—or in other words the culture vessel may be a bioreactor —, wherein the bioreactor preferably is a stirred bioreactor, a rocking motion bioreactor and/or a multi parallel bioreactor. As used herein, the terms “reactor” and “bioreactor”, which can be used interchangeably, refer to a closed culture vessel configured to provide a dynamic fluid environment for cell cultivation. The bioreactor may be stirred and/or agitated. Examples of agitated reactors include, but are not limited to, stirred tank bioreactors, wave-mixed/rocking bioreactors, up and down agitation bioreactors (i.e., agitation reactor comprising piston action), spinner flasks, shaker flasks, shaken bioreactors, paddle mixers, vertical wheel bioreactors. An agitated reactor may be configured to house a cell culture volume of between about 2 mL-20,000 L. Preferred bioreactors may have a volume of up to 50 L. An exemplary bioreactor suitable for the method of the present invention is the UniVessel bioreactor available from Sartorius Stedim Biotech. The bioreactor can be a stainless steel or a single use bioreactor. The bioreactor can consist of a single vessel or can comprise several bioreactors in parallel. The single use bioreactor can be manufactured from glass or plastic. The single use bioreactor can be a stirred tank bioreactor or a rocking motion bioreactor. Examples: Sartorius STR, RM, UniVessel. The pH of the culture medium may be controlled by the bioreactor, preferably controlled by CO₂ supply, and may be held in a range of 6.6 to 7.6, preferably at about 7.4.

The bioreactor may be a stirred bioreactor (STR). STRs are, e.g., available from Sartorius Stedim Biotech and include, but are not limited to, BIOSTAT® A/B/B-DCU/Cplus/D-DCU. The bioreactor may be a rocking motion bioreactor (RM). RMs are, e.g., available from Sartorius Stedim Biotech and include, but are not limited to, BIOSTAT® RM and BIOSTAT® RM TX. The bioreactor may be a multi parallel bioreactor that is.

In some embodiments, the volume of the culture vessel in the bioreactor is from about 50 mL to about 20,000 L. In some embodiments, the volume of the culture vessel in the bioreactor is from about 50 mL to about 2,000 L. In some embodiments, the volume of the culture vessel in the bioreactor is from about 50 mL to about 200 L. In some embodiments, the volume of the culture vessel in the bioreactor is from about 50 mL to about 100 L. In some embodiments, the volume of the culture vessel in the bioreactor is from about 50 mL to about 50 L. In some embodiments, the volume of the culture vessel in the bioreactor is from about 50 mL to about 20 L. In some embodiments, the volume of the culture vessel in the bioreactor is from about 50 mL to about 10 L. In some embodiments, the volume of the culture vessel in the bioreactor is from about 50 mL to about 1 L. In some embodiments, the volume of the culture vessel in the bioreactor is from about 100 mL to about 10 L. In some embodiments, the volume of the culture vessel in the bioreactor is from about 100 mL to about 5 L. In some embodiments, the volume of the culture vessel in the bioreactor is from about 150 mL to about 1 L. In some embodiments, the volume of the culture vessel in the bioreactor is from about 1 L to about 1,000 L.

One advantage of the present invention is that the cells can be grown in a closed system, i.e. there is no need of manual interaction or any interaction or manipulation of the cells outside their culture medium. Accordingly, the medium exchange may be performed inside the culture vessel or a bioreactor. Thereby, the cell aggregates can be kept in suspension culture in the culture vessel/bioreactor while a continuous medium exchange is performed while manual interaction with the suspension culture can be minimized or avoided.

It is however also possible that the medium exchange takes place outside of the culture vessel (of a bioreactor) while still a closed system without the need of human interaction is employed. Here, the rotating mesh is placed in a device housing that is outside the bioreactor. One outlet of the bioreactor is coupled to the “inside” section of the device housing to allow a liquid flow of the suspension culture comprising cell aggregates into the device housing. In addition, one outlet from the “inside” section of the device housing is coupled to the culture vessel (of a bioreactor). The used medium is perfused through the rotating mesh and discarded via a separate outlet. The discarded medium can be replaced by fresh medium in the device housing or in the bioreactor itself. Accordingly, the medium exchange may be performed outside of a bioreactor, preferably wherein the device housing the rotating mesh is fluidly coupled with the bioreactor to form a closed system.

A “growth medium”, “culture medium” or simply “medium” as used herein is a liquid designed to support the growth of microorganisms, cells, or small plants. Different types of media are used for growing different types of cells. A person skilled in the art is able to determine which culture medium is optimal for a specific cell type. The stem cells cultured in suspension (in the bioreactor) are cultured in a culture medium. Culture media that allow the expansion of the stem cells, i.e. defines some of the “conditions that allow proliferation of the stem cells”, are known to a person skilled in the art and include, but are not limited to, IPS-Brew, iPS-Brew XF, E8, StemFlex, mTeSR1, PluriSTEM, StemMACS, TeSRTM2, Corning NutriStem hPSC XF Medium, Essential 8 Medium (ThermoFisher Scientific), StemFit Basic02 (Ajinomoto Co. Inc), to name only a few. In one illustrative example, the culture medium is IPS-Brew that is available in GMP grade from Miltenyi Biotec, Germany. Another condition that determines whether the conditions are suitable for the expansion of the stem cells includes temperature. Accordingly, wherein the temperature of the culture medium is about 30 to about 50° C., about 30 to about 43° C., about 35 to about 40° C., about 36 to about 38° C., about 30 to about 37° C., about 32 to about 36° C., or about 37° C., preferably 37° C. Further conditions that allow proliferation of the stem cells may include pH of the medium, oxygen supply and/or stirring rate.

The method of changing culture medium disclosed herein can be used to replace the used media with the same (type of) medium or can also be used to perform a medium exchange to a different medium, e.g. for inducing differentiation or expression of a protein of interest under the control of an inducible promoter.

As outlined herein, the cells in the suspension culture are preferably not sedimented but distributed in the culture medium. Accordingly, the suspension culture preferably is stirred. Continuous stirring may lead to an essentially homogenous distribution of the cells in the culture medium/suspension culture and may help stem cells, in particular PSCs such as iPSCs to maintain their pluripotency. Accordingly, the cells preferably are essentially homogenously distributed in the culture medium.

The method of the present invention can generally be used for any cell that can be cultivated in cell culture, i.e. also for adherent cell culture. Advantageously, the method is used for changing culture medium of a suspension culture, in which the separation of the cells from the culture medium is of essence. In this context, “suspended in the culture medium” refers to cells that are cultured in suspension regardless if they actually are suspension cells or not. Thus, the method of the present invention can also be used for adherent cells, if they are suspended in the culture medium. Accordingly, the cells may by adherent cells that are cultured in suspension.

Adherent cells that are cultured in suspension, i.e. cannot attach to the culture vessel, may form cell aggregates. This also applies to the stem cells cultured in the uses and methods described herein. As used herein, the terms “aggregate” and “cell aggregate”, which may be used interchangeably, refer to a plurality of cells such as (induced) pluripotent stem cells, in which an association between the cells is caused by cell-cell interaction (e.g., by biologic attachments to one another). Biological attachment may be, for example, through surface proteins, such integrins, immunoglobulins, cadherins, selectins, or other cell adhesion molecules. For example, cells may spontaneously associate in suspension and form cell-cell attachments (e.g., self-assembly), thereby forming aggregates. In some embodiments, a cell aggregate may be substantially homogeneous (i.e., mostly containing cells of the same type). In other embodiments, a cell aggregate may be heterogeneous, (i.e., containing cells of more than one type).

The method of the invention is suitable for cell aggregates. The cell aggregates may vary in size. In case of stem cells, the cells form cell aggregates, which typically have an average diameter of about 50 to about 150 μm such as about 100 μm 1 day after seeding (see also Example 2). The initial average diameter accordingly preferably is about 50 to about 150 μm, more preferably about 100 μm. After four days, the cell aggregates typically have an average diameter of about 200 to about 220 μm (see also Example 2). The final average diameter of the cell aggregates thus is preferably about 200 to about 200 μm. At this diameter, the stem cell aggregates ideally are dissociated, since diameters exceeding about 300 μm may result in cell necrosis due to the limited nutrient and gas diffusion into the tissue/aggregate center. Eventually, uncontrolled differentiation—particularly in large stem cell aggregates—might also occur. Accordingly, the cell aggregates are preferably dissociated when having average diameter of about 180 to about 250 μm, preferably about 200 to about 220 μm, ideally about 200 μm. Accordingly, the cell aggregates may have an average diameter between about 50 and about 300 μm, between about 80 and about 250 μm, between about 100 and about 220 μm or between about 100 μm to about 200 μm. Preferably, the average diameter of the cell aggregates is between about 50 μm to about 220 μm, more preferably between about 100 μm to about 200 μm. The cell aggregates may have an average diameter between about 50 and 800 μm, between about 150 and 800 μm, of at least about 800 μm, of at least about 600 μm, of at least about 500 μm, of at least about 400 μm, of at least about 300 μm, of at least about 200 μm, of at least about 150 μm, between about 300 and 500 μm, between about 150 and 300 μm, between about 50 and 150 μm, between about 80 to 100 μm, between about 180 to 250 μm or between about 200 to 250 μm

The cells may be any cells that can be cultivated in suspension, preferably the cells are stem cells. In multicellular organisms, stem cells are undifferentiated or partially differentiated cells that can differentiate into various types of cells and proliferate indefinitely to produce more of the same stem cell. They are usually distinguished from progenitor cells, which cannot divide indefinitely, and precursor or blast cells, which are usually committed to differentiating into one cell type. The term stem cells thus encompasses pluripotent stem cells but also multipotent (can differentiate into a number of cell types, but only those of a closely related family of cells), oligopotent stem cells (can differentiate into only a few cell types, such as lymphoid or myeloid stem cells) or unipotent stem cells such as satellite cells. Examples of stem cells include, but are not limited to, pluripotent stem cells, cord blood stem cells, mesenchymal stem cell and/or hematopoietic stem cells, preferably pluripotent stem cells. Particularly preferred are induced pluripotent stem cells (iPSCs). In the context of the present invention, the stem cells may also relate to cells derived from stem cells, in particular cells derived from (i) PSCs. “Cells derived from stem cells” relate to differentiated cells or cells differentiated into a specific cell type that are no longer capable of differentiating in any cell type of the body. Said cells derived from stem cells relate to cells, which are derived from the (pluripotent) stem cells used in the methods and uses of the invention and thus preferably do not include naturally occurring differentiated cells. Methods for the differentiation into different cell types starting from the stem cells such as PSCs are known to a person skilled in the art. “Cells derived from stem cells” may relate to heart cells and/or tissue, liver cells and/or tissue, kidney cells and/or tissue, brain cells and/or tissue, pancreatic cells and/or tissue, lung cells and/or tissue, skeletal muscle cells and/or tissue, gastrointestinal cells and/or tissue, neuronal cells and/or tissue, skin cells and/or tissue, bone cells and/or tissue, bone marrow, fat cells and/or tissue, connective cells and/or tissue, retinal cells and/or tissue, blood vessel cells and/or tissue, stromal cells or cardiomyocytes. Methods for generating heart tissue are known from WO 2015/025030 and WO 2015/040142. The cells may also be differentiated in the bioreactor or also outside of the bioreactor, e.g. to cardiomyocytes or stromal cells. These differentiated cells may also be cultured in a bioreactor making use of the method of the invention. Cells obtained from a tissue or an organ may be obtained from heart cells and/or tissue, liver cells and/or tissue, kidney cells and/or tissue, brain cells and/or tissue, pancreatic cells and/or tissue, lung cells and/or tissue, skeletal muscle cells and/or tissue, gastrointestinal cells and/or tissue, neuronal cells and/or tissue, skin cells and/or tissue, bone cells and/or tissue, bone marrow, fat cells and/or tissue, connective cells and/or tissue, retinal cells and/or tissue, blood vessel cells and/or tissue, stromal cells or cardiomyocytes.

The cells may be cells of a mammal, such as a human, a dog, a mouse, a rat, a pig, a non-human primate such as Rhesus macaque, baboon, cynomolgus macaque or common marmoset to name only a few illustrative examples. Preferably, the cells are human.

The term “pluripotent stem cell” (PSC) as used herein refers to cells that are able to differentiate into every cell type of the body. As such, pluripotent stem cells offer the unique opportunity to be differentiated into essentially any tissue or organ. Currently, the most utilized pluripotent cells are embryonic stem cells (ESC) or induced pluripotent stem cells (iPSC). Further examples of pluripotent stem cells include parthenogenetic stem cells (pPSC) or nuclear transfer derived PSCs (ntPSC). Human ESC-lines were first established by Thomson and coworkers (Thomson et al. (1998), Science 282:1145-1147). Human ESC research recently enabled the development of a new technology to reprogram cells of the body into an ES-like cell. This technology was pioneered by Yamanaka and coworkers in 2006 and 2007 (Takahashi & Yamanaka (2006), Cell, 126:663-676 and Takahashi et al. (2007), Cell, 131(5):861-72).

Resulting induced pluripotent cells (iPSC) show a very similar behavior as ESC and, importantly, are also able to differentiate into every cell of the body. Thus, in one embodiment, the term iPSCs comprises ESC. In the context of the present invention, these pluripotent stem cells are however preferably not produced using a process which involves modifying the germ line genetic identity of human beings or which involves use of a human embryo for industrial or commercial purposes. Preferably, the pluripotent stem cells are of primate origin, more preferably human.

Suitable induced PSCs, can for example, be obtained from the NIH human embryonic stem cell registry, the European Bank of Induced Pluripotent Stem Cells (EBiSC), the Stem Cell Repository of the German Center for Cardiovascular Research (DZHK), the Human Pluripotent Stem Cell Registry (hPSCreg), or ATCC, to name only a few sources. Induced pluripotent stem cells are also available for commercial use, for example, from the NINDS Human Sequence and Cell Repository (https://stemcells.nindsgenetics.org) which is operated by the U.S. National Institute of Neurological Disorders and Stroke (NINDS) and distributes human cell resources broadly to academic and industry researchers. One illustrative example of a suitable cell line that can be used in the present invention is the cell line TC-1133, an induced (unedited) pluripotent stem cell that has been derived from a cord blood stem cell. This cell line is, e.g. directly available from NINDS, USA. Preferably, TC-1133 is GMP-compliant. Further exemplary iPSC cell lines that can be used in the present invention, include but are not limited to, the Human Episomal iPSC Line of Gibco™ (order number A18945, Thermo Fisher Scientific), or the iPSC cell lines ATCC ACS-1004, ATCC ACS-1021, ATCC ACS-1025, ATCC ACS-1027 or ATCC ACS-1030 available from ATTC. Alternatively, any person skilled in the art of reprogramming can easily generate suitable iPSC lines by known protocols such as the one described by Okita et al, “A more efficient method to generate integration-free human iPS cells” Nature Methods, Vol. 8 No. 5, May 2011, pages 409-411 or by Lu et al “A defined xeno-free and feeder-free culture system for the derivation, expansion and direct differentiation of transgene-free patient-specific induced pluripotent stem cells”, Biomaterials 35 (2014) 2816e2826.

The stem cells may be selected from the group consisting of TC-1133, the Human Episomal iPSC Line of Gibco, ATCC ACS-1004, ATCC ACS-1021, ATCC ACS-1025, ATCC ACS-1027, ATCC ACS-1030.

As explained herein, the (induced) pluripotent stem cell that is used in the present invention can be derived from any suitable cell type (for example, from a stem cell such as a mesenchymal stem cell, or an epithelial stem cell or a differentiated cells such as fibroblasts) and from any suitable source (bodily fluid or tissue). Examples of such sources (body fluids or tissue) include cord blood, skin, gingiva, urine, blood, bone marrow, any compartment of the umbilical cord (for example, the amniotic membrane of umbilical cord or Wharton's jelly), the cord-placenta junction, placenta or adipose tissue, to name only a few. In one illustrative example, is the isolation of CD34-positive cells from umbilical cord blood for example by magnetic cell sorting using antibodies specifically directed against CD34 followed by reprogramming as described in Chou et al. (2011), Cell Research, 21:518-529. Baghbaderani et al. (2015), Stem Cell Reports, 5(4):647-659 show that the process of iPSC generation can be in compliance with the regulations of good manufacturing practice to generate cell line N D50039.

Accordingly, the stem cell preferably fulfils the requirements of the good manufacturing practice.

The present invention further relates to the use of a rotating mesh as defined herein for medium exchange in a suspension culture, the suspension culture comprising cell aggregates suspended in the culture medium, wherein the cells are stem cells.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “less than” or in turn “more than” does not include the concrete number.

For example, less than 20 means less than the number indicated. Similarly, more than or greater than means more than or greater than the indicated number, e.g. more than 80% means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

As used herein the terms “about”, “approximately” or “essentially” mean within 20%, preferably within 15%, preferably within 10%, and more preferably within 5% of a given value or range. It also includes the concrete number, i.e. “about 20” includes the number of 20.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

### EXAMPLES

An even better understanding of the present invention and of its advantages will be evident from the following examples, offered for illustrative purposes only. The examples are not intended to limit the scope of the present invention in any way.

### Example 1: Application of a Spinfilter Allows Automated Medium Exchange without Altering the Morphology of the PSC Aggregates

To following material and equipment (see Table 1) was used according to the manufacturer's instructions:

TABLE 1
Materials used in Example 1.

| Material and equipment | Detail |
|---|---|
| iPSCs | TC1133: TC1133 is a human iPS cell line |
| | that was generated by Lonza under cGMP- |
| | compliant conditions (Baghbaderani et al., |
| | 2015, 2016) |
| Bioreactor | UniVessel 0.5 L (Sartorius) equipped |
| | with a 10 μm spinfilter (Sartorius) |
| Bioreactor controller | Biostat B - DCU II (Sartorius) |
| Cell counter | Nucleocounter 200 (Chemometec) |

The cell-only aggregate suspension culture was performed as described in the following. Cells were seeded at a concentration of 2.5×10⁺⁶ cells/ml and were cultured in StemMACS iPS-Brew XF, Basal Medium. Medium exchange by perfusion through the spinfilter was started at day 2. The following Table 2 shows the culture parameters.

TABLE 2
Culture parameters of Example 1.

| Parameter | UniVessel 0.5 L | |
|---|---|---|
| Temperature | 37° | C. |
| pH | 7.4 | |
| Oxygen concentration | 23.8% air saturation | |
| Stirring speed | 85-140 | rpm |
| Stirring direction | Downwards | |
| Blade angle of impeller | 30-50° (preferably 45°) | |
| Cultivation volume | 150-500 | mL |
| Seeding density | 2.5 × 10⁵ | cells/mL |
| Medium exchange volume | 62-100% per day with spinfilter | |
| Beginning of medium exchange | Day | 2 |

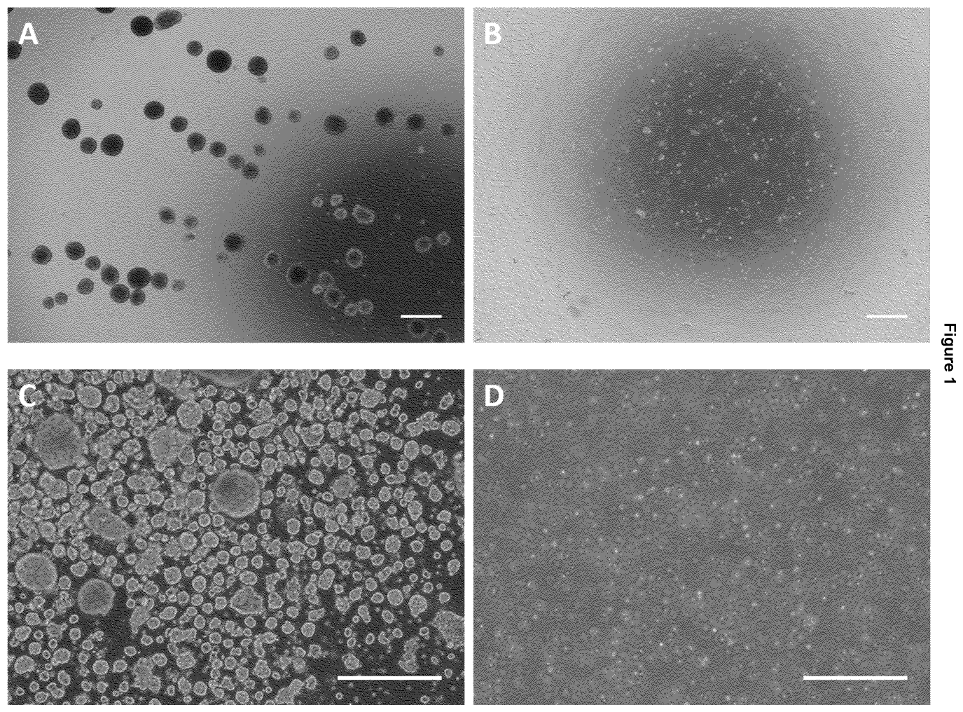

The spinfilter successfully retains iPSC aggregates with a size of ˜70-300 μm when used at stirring speeds of 90-140 rpm and 5-100% pump rate, corresponding to about 0.1 to 2.2 mL/min. Perfusion medium exchange of 60-100% medium exchange rate per day is performed successfully at a culture volume of 300-500 mL. The spent medium, which is removed using a spinfilter, contains no iPSC aggregates but only single cells and debris (FIGS. 1 B and D) while the iPSC aggregates in the culture have a typical morphology (FIGS. 1A and C).

In sum, the Inventors could surprisingly show that the application of a rotating mesh, here exemplarily a spinfilter, does not harm the iPSC aggregates and allows a continuous perfusion culture.

### Example 2: Application of a Spinfilter has No Influence on Quality of PSC Aggregates

The inventors repeated the experiment shown in Example 1 with a 0.5 L and a 2 L UniVessel to further underline the applicability of spinfilters for medium exchange of PSC cell aggregate suspension culture, also in respect of aggregate size, expansion rate and pluripotency.

Materials and methods correspond to Example 1 with the following culture parameters outlined in Table 3. iPSCs were cultured in “vessel 2” (internal designation for a Sartorius UniVessel 2 L), which and in “vessel 3” (internal designation for a Sartorius UniVessel 0.5 L).

TABLE 3
Culture parameters of Example 2.

| | UniVessel 0.5 L | | UniVessel 2 L | |
|---|---|---|---|---|
| Parameter | (“vessel 3”) | | (“vessel 2”) | |
| Temperature | 37° | C. | 37° | C. |
| pH | 7.4 | | 7.4 | |
| Oxygen concentration | 23.8% air | | 23.8% air | |
| | saturation | | saturation | |
| Stirring speed | 100 | rpm | 70 | rpm |
| Stirring direction | Downwards | | Downwards | |
| Blade angle of impeller | 45° | | 45° | |
| Cultivation volume | 330 | ml | 500 | mL |
| Seeding density | 2.5 × 10⁵ | cells/mL | 2.5 × 10⁵ | cells/mL |
| Medium exchange | 62% per day | | 50% per day | |
| volume | with spinfilter | | with spinfilter | |
| Beginning of medium | Day | 2 | Day | 2 |
| exchange | | | | |

### Aggregate Size

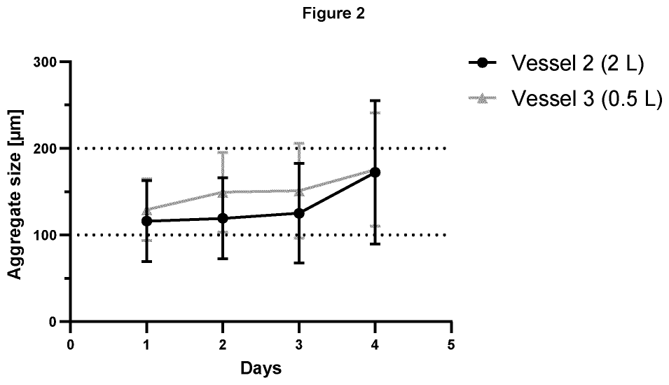

The aggregates in the UniVessel 0.5 L were large on day 1 of passage 0 with 129 μm (FIG. 2Fehler! Verweisquelle konnte nicht gefunden werden.). Aggregates in the UniVessel 2 L on day 1 of passage 0 were smaller than the ones in the 0.5 L vessel. On day 4 of passage 0, the aggregates of both vessels were comparable in size.

### Expansion Rate

The expansion rate after 4 days of culture in passage 0 was about 8-fold in both the 0.5 L and 2 L UniVessel (Table 4).

TABLE 4
Expansion rate of passage 0.

| | Day 0 | | Day 4 | | |
|---|---|---|---|---|---|
| | Cell | | Cell | | Expansion |
| | concentration | Cell | concentration | Cell | rate [fold |
| Sample | [cells/mL] | number | [cells/mL] | number | change] |
| UniVessel Vessel 3 (0.5 L) | 2.34E+05 | 1.17E+08 | 1.35E+06 | 9.68E+08 | 8.28× |
| UniVessel Vessel 2 (2 L) | 2.26E+05 | 7.23E+07 | 1.93E+06 | 6.35E+08 | 8.78× |

### Pluripotency

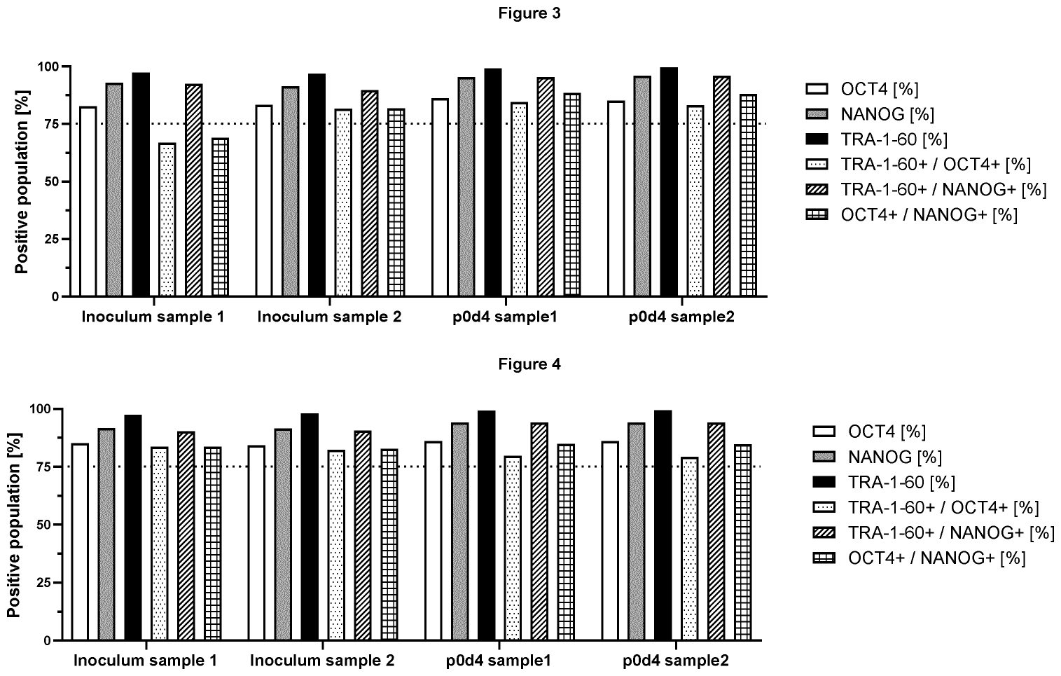

The expression of pluripotency-related genes was high in the inoculum of both vessels (FIGS. 3 and 4). iPSCs of both the 2 L and the 0.5 L UniVessels showed a high expression of pluripotency-related markers at day 4 of passage 0. The expression in iPSCs in suspension was comparable to the expression in the inoculum.

### Analysis

In Example 2, iPSCs were cultured in two UniVessels of different sizes (0.5 L and 2 L). In both Vessels iPSCs of good quality were obtained at day 4 of passage 0. Therefore, using the method of the present invention leads to a more desirable quality at desirable growth rates and relevant aggregate sizes.

### REFERENCES

Baghbaderani, B. A., Tian, X., Neo, B. H., Burkall, A., Dimezzo, T., Sierra, G., Zeng, X., Warren,
K., Kovarcik, D. P., Fellner, T., et al. (2015). cGMP-Manufactured Human Induced Pluripotent Stem Cells Are Available for Pre-clinical and Clinical Applications. Stem Cell Reports 5, 647-659.
Baghbaderani, B. A., Syama, A., Sivapatham, R., Pei, Y., Mukherjee, O., Fellner, T., Zeng, X., and Rao, M. S. (2016). Detailed Characterization of Human Induced Pluripotent Stem Cells Manufactured for Therapeutic Applications. Stem Cell Rev and Rep 12, 394-420.
Baptista, R. P., Fluri, D. A., and Zandstra, P. W. (2013). High density continuous production of murine pluripotent cells in an acoustic perfused bioreactor at different oxygen concentrations. Biotechnology and Bioengineering 110, 648-655.
Chen, V. C., Couture, S. M., Ye, J., Lin, Z., Hua, G., Huang, H.-I. P., Wu, J., Hsu, D., Carpenter, M. K., and Couture, L. A. (2012). Scalable GMP compliant suspension culture system for human ES cells. Stem Cell Research 8, 388-402.
Fernandes-Platzgummer, A., Diogo, M. M., Lobato da Silva, C., and Cabral, J. M. S. (2014). Maximizing mouse embryonic stem cell production in a stirred tank reactor by controlling dissolved oxygen concentration and continuous perfusion operation. Biochemical Engineering Journal 82, 81-90.
Halloin, C., Coffee, M., Manstein, F., and Zweigerdt, R. (2019). Production of Cardiomyocytes from Human Pluripotent Stem Cells by Bioreactor Technologies. In Cell-Based Assays Using IPSCs for Drug Development and Testing, C.-F. Mandenius, and J. A. Ross, eds. (New York, NY: Springer New York), pp. 55-70.
Hemmi, N., Tohyama, S., Nakajima, K., Kanazawa, H., Suzuki, T., Hattori, F., Seki, T., Kishino,
Y., Hirano, A., Okada, M., et al. (2014). A Massive Suspension Culture System With Metabolic Purification for Human Pluripotent Stem Cell-Derived Cardiomyocytes. STEM CELLS Translational Medicine 3, 1473-1483.
Jiang, Y., Langenberg, K., Borgdorff, V., Dunska, M., Post, R., Bartulos, O., Doornbos, M., Braam, S., Reijerkerk, A., and Rasche, U. (2019). Controlled, Large-Scale Manufacturing of hiPSC-Derived Cardiomyocytes in Stirred-Tank Bioreactors. 12.
Kempf, H., Kropp, C., Olmer, R., Martin, U., and Zweigerdt, R. (2015). Cardiac differentiation of human pluripotent stem cells in scalable suspension culture. Nature Protocols 10, 1345-1361.
Kropp, C., Kempf, H., Halloin, C., Robles-Diaz, D., Franke, A., Scheper, T., Kinast, K., Knorpp, T., Joos, T. O., Haverich, A., et al. (2016). Impact of Feeding Strategies on the Scalable Expansion of Human Pluripotent Stem Cells in Single-Use Stirred Tank Bioreactors. STEM CELLS Translational Medicine 5, 1289-1301.
Kropp, C., Massai, D., and Zweigerdt, R. (2017). Progress and challenges in large-scale expansion of human pluripotent stem cells. Process Biochemistry 59, 244-254.
Lipsitz, Yonatan Y.; Tonge, Peter D.; Zandstra, Peter W. (2018): Chemically controlled aggregation of pluripotent stem cells. In: Biotechnology and bioengineering 115 (8), S. 2061-2066. DOI: 10.1002/bit.26719.
Serra, M., Brito, C., Sousa, M. F. Q., Jensen, J., Tostões, R., Clemente, J., Strehl, R., Hyllner, J., Carrondo, M. J. T., and Alves, P. M. (2010). Improving expansion of pluripotent human embryonic stem cells in perfused bioreactors through oxygen control. Journal of Biotechnology 148, 208-215.
Voisard, D., Meuwly, F., Ruffieux, P.-A., Baer, G., and Kadouri, A. (2003). Potential of cell retention techniques for large-scale high-density perfusion culture of suspended mammalian cells. Biotechnology and Bioengineering 82, 751-765.

1. A method of expanding stem cells, wherein the stem cells are comprised in cell aggregates in a suspension culture, the method comprising:
(i) culturing the stem cells under conditions that allow proliferation of the stem cells; and
(ii) performing medium exchange by perfusion through a rotating mesh.

2. A method of changing culture medium of a suspension culture, the suspension culture comprising cell aggregates of stem cells suspended in the culture medium, the method comprising:
(i) performing medium exchange by perfusion through a rotating mesh; and
(ii) optionally replacing the medium removed through the rotating mesh with fresh medium.

3. The method of any one of the preceding claims, wherein the stem cells are cultured in a bioreactor, wherein the bioreactor preferably is a stirred bioreactor, a rocking motion bioreactor and/or a multi parallel bioreactor.

4. The method of any one of the preceding claims, wherein the medium exchange is performed inside a bioreactor.

5. The method of any one of the preceding claims, wherein the rotating mesh is a spin-filter, optionally wherein the spin-filter is attached to the stirrer or stirring rod of a bioreactor.

6. The method of any one of claim 1-3 or 5, wherein the medium exchange is performed outside of a bioreactor, preferably wherein the device housing the rotating mesh is fluidly coupled with the bioreactor to form a closed system.

7. The method of any one of the preceding claims, wherein the rotating mesh has a pore size of about 1 μm to about 50 μm, of about 5 μm to about 50 μm, of about 10 μm to about 50 μm, of about 5 μm to about 40 μm, about 5 μm to about 30 μm, about 5 μm to about 20 μm, or about 5 μm to about 15 μm, preferably about 10 μm.

8. The method of any one of the preceding claims, wherein the cell aggregates have an average diameter between about 50 and about 300 μm, between about 80 and about 250 μm, between about 100 and about 220 μm or between about 100 μm to about 200 μm.

9. The method of any one of the preceding claims, wherein the stem cells are pluripotent stem cells, cord blood stem cells, mesenchymal stem cell and/or hematopoietic stem cells; and/or cells derived from stem cells, wherein the pluripotent stem cells preferably are induced pluripotent stem cells (iPSC), embryonic stem cells (ESC), parthenogenetic stem cells (pPSC) or nuclear transfer derived PSCs (ntPSC), most preferably iPSCs.

10. The method of any one of the preceding claims, wherein the stem cells are selected from the group consisting of TC-1133, the Human Episomal iPSC Line of Gibco ATCC ACS-1004, ATCC ACS-1021, ATCC ACS-1025, ATCC ACS-1027, ATCC ACS-1030.

11. Use of a rotating mesh for medium exchange in a suspension culture, the suspension culture comprising cell aggregates suspended in the culture medium, wherein the cells are stem cells.

12. The use of claim 11, wherein the stem cells are pluripotent stem cells, cord blood stem cells, mesenchymal stem cell and/or hematopoietic stem cells; and/or cells derived from stem cells, wherein the pluripotent stem cells preferably are induced pluripotent stem cells (iPSC), embryonic stem cells (ESC), parthenogenetic stem cells (pPSC) or nuclear transfer derived PSCs (ntPSC), most preferably iPSCs.

13. The use of claim 11 or 12, wherein the stem cells are selected from the group consisting of TC-1133, the Human Episomal iPSC Line of Gibco ATCC ACS-1004, ATCC ACS-1021, ATCC ACS-1025, ATCC ACS-1027, ATCC ACS-1030.

14. The use of any one of claims 11-13, wherein the cell aggregates preferably have an average diameter average diameter between about 50 and about 300 μm, between about 80 and about 250 μm, between about 100 and about 220 μm or between about 100 μm to about 200 μm.

15. The use of claim 11, wherein the rotating mesh is a spin-filter, optionally wherein the spin-filter is attached to the stirrer or stirring rod of a bioreactor.

16. The use of claim 11 or 15, wherein the rotating mesh has a pore size of about 1 μm to about 50 μm, of about 5 μm to about 50 μm, of about 10 μm to about 50 μm, of about 5 μm to about 40 μm, about 5 μm to about 30 μm, about 5 μm to about 20 μm, or about 5 μm to about 15 μm, preferably about 10 μm.
